(19) United States
(12) Patent Application Publication
(10) Pub. No.: US 2022/0011465 A1
(43) Pub. Date: Jan. 13, 2022

(54) Systems and Methods for Hydrocarbon Reservoir Divided Model Generation and Development

(71) Applicant: Saudi Arabian Oil Company, Dhahran (SA)

(72) Inventor: Kenneth Richard Kibodeaux, Pearland, TX (US)

(21) Appl. No.: 17/356,025

(22) Filed: Jun. 23, 2021

Related U.S. Application Data
(60) Provisional application No. 63/048,710, filed on Jul. 7, 2020.

(51) Int. Cl. G01V 99/00; E21B 44/00; G01N 33/24; G01N 23/2251; G01N 23/2255; G06F 30/20

(52) U.S. Cl. CPC G01V 99/005; E21B 44/00; G01N 33/24; E21B 49/02; G01N 23/2255; G06F 30/20; E21B 2200/20; G01N 23/2251

(57) ABSTRACT

Provided are techniques for developing a hydrocarbon reservoir that include: determining a reservoir model of a hydrocarbon reservoir that includes columns of gridblocks that represent a vertical segment of the reservoir; acquiring nano-images of a rock sample of the reservoir; determining, based on the nano-images, properties of an inorganic pore network and an organic pore network of the rock sample; generating a divided reservoir model of the reservoir that represents the inorganic and organic pore networks of the reservoir, including: for each of the columns of gridblocks, dividing each of the gridblocks of the column into: a water-wet gridblock associated with the properties of the inorganic pore network determined based on the nano-images; and an oil-wet gridblock associated with the properties of the organic pore network determined based on the nano-images; and generating, using the divided reservoir model, a simulation of the hydrocarbon reservoir.

### RELATED APPLICATIONS

This application claims the benefit of U.S. Provisional Patent Application No. 63/048,710 filed on Jul. 7, 2020 and titled “SYSTEMS AND METHODS FOR HYDROCARBON RESERVOIR DIVIDED MODEL GENERATION AND DEVELOPMENT” which is hereby incorporated by reference in its entirety.

### FIELD

Embodiments relate generally to developing hydrocarbon reservoirs, and more particularly to hydrocarbon reservoir simulation and development.

### BACKGROUND

A rock formation that resides underground is often referred to as a “subsurface formation.” A porous or fractured rock formation that contains, or that is expected to contain, a subsurface accumulation of hydrocarbons, such as oil and gas, is often referred to as a “hydrocarbon reservoir.” In many instances, hydrocarbons are extracted (or “produced”) from a hydrocarbon reservoir by way of a well. A well generally includes a wellbore (or “borehole”) that is drilled into the Earth. A hydrocarbon well may extend into a hydrocarbon reservoir to, for example, facilitate extraction of hydrocarbons from the reservoir, the injection of fluids into the reservoir, or the evaluation and monitoring of the reservoir.

Exploration for and production of hydrocarbons can involve multiple complex phases that are employed to optimize extraction of the hydrocarbons. For example, a reservoir operator may spend time and effort assessing a hydrocarbon reservoir to identify economical and environmentally responsible ways to extract hydrocarbons from the reservoir. This can include identifying where hydrocarbons are located in the reservoir and generating a field development plan (FDP) that outlines procedures for extracting hydrocarbons from the reservoir. An FDP may specify, for example, locations, trajectories and operational parameters of production wells, injection wells and monitoring wells drilled into the reservoir. In many instances, operators rely on simulations to characterize a reservoir and, then, develop an FDP based on the characterization. For example, an operator may run simulations of a reservoir to determine where fluids, such as water and hydrocarbons, are located in the reservoir and the fluids are expected to move within the reservoir and, then, use the results of the simulations to generate or adjust an FDP for the reservoir. In many instances, simulations are run and FDPs are updated over the course of the development of a reservoir. For example, initial simulations may be run to determine locations and operating parameters for wells before they are drilled, and follow-up simulations may be run to determine updated operating parameters for the wells already drilled and locations and operating parameters for additional wells to be drilled. Accordingly, simulations can be an important aspect of developing a reservoir.

### SUMMARY

Successful development of a hydrocarbon reservoir often relies on the ability to provide accurate and timely simulations of the reservoir. For example, a reservoir operator may be able to identify movement of hydrocarbons and other substances, such as injected water, within a reservoir based on a simulation and develop the reservoir accordingly. This can include locating wells and adjusting well operating parameters, such as production and injection rates, to optimize extraction of hydrocarbons from the reservoir.

Simulation of a reservoir often involves modeling the reservoir with a three-dimensional (3D) grid of individual cells. The grid often takes the form of a structured 3D grid that includes 3D volumes, such as cubes (or “gridblocks”) arranged in rows and columns, representing the reservoir. Each of the cells of a model may represent a respective portion of the reservoir and be associated with corresponding properties of the portion of the reservoir represented by the cell. During a simulation of a reservoir, the cells of a grid may be processed in parallel by different computing processes. In some instances, a grid can include millions or billions of cells, and the simulation run can involve hundreds or thousands of processors working in parallel and exchanging information regarding the particular cells they are processing. Unfortunately, even with the implementation of a relatively large number of processors, simulations can still require a great deal of time to complete. In some instances simulations can take hours, days, weeks or months to complete. Thus, simulations can be costly from the perspective of computing resources and may introduce delays into developing and updating field development plans (FDPs) for a reservoir. Unfortunately, field operators will sometimes forgo simulations due to a lack of resources. Accordingly, efficient use of computing resources can be helpful in providing accurate and timely simulations that can assist with effective development of reservoirs.

Attempts to improve simulation performance, including attempts to improve simulation accuracy, speed or efficiency, often have shortcomings. For example, reducing the number of cells of a model may reduce accuracy of a corresponding simulation, and increasing the number of cells of a model may increase processing overhead of a corresponding simulation. Thus, generating a suitable model can include reducing or increasing cells in a model to find a balance between speed and accuracy. Within that balance, there are challenges associated with identifying properties of cells, determining which cells can and should be modified, and making any modifications in a manner that honors elements of the reservoir in a manner that is computationally possible and efficient.

In many instances, modeling unconventional rock, such as tight source rock, or other types of rock having mixed wettability values, is difficult. Tight source rock, such as unconventional shale, is rock that exhibits two distinct pore networks—an inorganic (or “water-wet”) pore network and an organic (or “oil-wet”) pore network. The presence of two different pore networks in rock can introduce complexities into associated modeling of the rock. For example, in the case of modeling tight source rock with a grid of cells, a corresponding model may need to account for variations of two distinct pore networks in each cell, as well account for varying degrees of connectivity between the two networks. Some techniques employ “multiple-continua” models seeking to capture behavior of two networks; however, those types of models tend to be complex and computationally burdensome.

In some embodiments, provided is an image-based modeling of rock having mixed wettability values, such as tight source rock. Modern imaging capabilities are capable of providing nano-scale details indicating the porosity, permeability, wettability, and mutual connectivity of inorganic and organic networks of mixed wettability rocks. For example, modern imaging techniques may provide resolution that enables deciphering details, such a pore sizes or other rock features, in the range of about 1-100 nanometers. Such nano-scale details can be employed to generate a divided model of a reservoir that distinctly models the inorganic and organic networks, as well as degrees of connectivity between the networks. In some embodiments, gridblocks of a reservoir model for rock having mixed wettability values, such as unconventional shale, are divided into two parts—(1) an “oil-wet” gridblock that represents an organic (or “oil-wet”) pore network, and (2) a “water-wet” gridblock that represents an inorganic (or “water-wet”) pore network. In some embodiments, the properties of the oil-wet gridblocks and water wet gridblocks (for example, the porosity, the permeability, and the wettability of both networks, as well as the mutual connectivity of the two networks) are derived from nano-images of the rock. Thus, the technique may enable linking nano-image-derived rock properties to field-scale recovery behaviors that are observed by way of simulation of a model containing “wettability” divided gridblocks. In some embodiments, modeling of a “mixed wettability” reservoir, such as a reservoir formed of unconventional shale or other tight source rocks, includes the following: (1) acquiring a sample of rock from the reservoir (for example, by way of a coring operation); (2) acquiring three-dimensional (3D) nano-images of the rock that capture an inner pore structure of the rock (for example, using Focused Ion Beam (FIB) and Scanning Electron Microscopy (SEM)); (3) determining (based on the nano-images of the rock) for each of (a) an inorganic pore network in the rock, and (b) an organic pore network in the rock, the following properties of the rock: porosity, permeability, pore-size distribution, wettability, degree of connectivity, amount of oil, amount of gas, and amount of water; (4) generating a “divided model” of the reservoir comprising, for each column of gridblocks of a model of the reservoir, dividing the gridblocks to generate two “divided block columns” that include the following: (a) a first/inorganic divided block column having gridblocks associated with corresponding properties of the inorganic pore network in the rock; (b) a second/organic divided block column having gridblocks associated with corresponding properties of the organic pore network in the rock; and (c) for each pair of neighboring grid blocks of the two divided block columns, a transmissibility multiplier that corresponds to the degree of connectivity determined; and (5) simulating the reservoir using the divided model.

Provided in some embodiments is a method of developing a hydrocarbon reservoir that includes: determining a reservoir model of a hydrocarbon reservoir that defines gridblocks that each represent a respective portion of the hydrocarbon reservoir and columns of the gridblocks that each represent a vertical segment of the hydrocarbon reservoir; acquiring nano-images of a rock sample acquired from the hydrocarbon reservoir; determining, based on the nano-images of the rock sample, properties of an inorganic pore network of the rock sample and properties of an organic pore network of the rock sample; generating a divided reservoir model of the hydrocarbon reservoir that is representative of the inorganic and organic pore networks of the hydrocarbon reservoir, the generating of the divided reservoir model including: for each of the columns of gridblocks, dividing each of the gridblocks of the column into: a water-wet gridblock associated with the properties of the inorganic pore network determined based on the nano-images of the rock sample; and an oil-wet gridblock associated with the properties of the organic pore network determined based on the nano-images of the rock sample; and generating, using the divided reservoir model of the hydrocarbon reservoir, a simulation of the hydrocarbon reservoir.

In some embodiments, acquiring nano-images of the rock sample includes conducting Focused Ion Beam (FIB) or Scanning Electron Microscopy (SEM) imaging of the rock sample to acquire FIB or SEM images of the rock sample. In certain embodiments, the reservoir model includes a three-dimensional grid of gridblocks that represents a portion of the hydrocarbon reservoir. In some embodiments, the method further includes determining that the portion of the hydrocarbon reservoir represented by the gridblocks has mixed wettability, where the generation of the divided model is conducted in response to determining that the portion of the hydrocarbon reservoir represented by the gridblocks has mixed wettability. In certain embodiments, the method further includes, for each pair of a water-wet gridblock and an oil-wet gridblock generated from a gridblock, determining a transmissibility multiplier that corresponds to a degree of connectivity between the water-wet gridblock and the oil-wet gridblock, where the divided reservoir model defines the transmissibility multiplier for each pair of a water-wet gridblock and an oil-wet gridblock. In certain embodiments, the method further includes generating, based on the simulation of the hydrocarbon reservoir, a field development plan (FDP) for the hydrocarbon reservoir. In some embodiments, the method further includes: identifying, based on the simulation of the hydrocarbon reservoir, well drilling parameters; and drilling, based on the well drilling parameters, a well in the hydrocarbon reservoir. In some embodiments, the method further includes: identifying, based on the simulation of the hydrocarbon reservoir, well operating parameters; and operating, based on the well operating parameters, a well in the hydrocarbon reservoir.

Provided in some embodiments is a non-transitory computer readable storage medium including program instructions stored thereon that are executable by a processor to perform the following operations for developing a hydrocarbon reservoir: determining a reservoir model of a hydrocarbon reservoir that defines gridblocks that each represent a respective portion of the hydrocarbon reservoir and columns of the gridblocks that each represent a vertical segment of the hydrocarbon reservoir; acquiring nano-images of a rock sample acquired from the hydrocarbon reservoir; determining, based on the nano-images of the rock sample, properties of an inorganic pore network of the rock sample and properties of an organic pore network of the rock sample; generating a divided reservoir model of the hydrocarbon reservoir that is representative of the inorganic and organic pore networks of the hydrocarbon reservoir, the generating of the divided reservoir model including: for each of the columns of gridblocks, dividing each of the gridblocks of the column into: a water-wet gridblock associated with the properties of the inorganic pore network determined based on the nano-images of the rock sample; and an oil-wet gridblock associated with the properties of the organic pore network determined based on the nano-images of the rock sample; and generating, using the divided reservoir model of the hydrocarbon reservoir, a simulation of the hydrocarbon reservoir.

In some embodiments, acquiring nano-images of the rock sample includes conducting Focused Ion Beam (FIB) or Scanning Electron Microscopy (SEM) imaging of the rock sample to acquire FIB or SEM images of the rock sample. In certain embodiments, the reservoir model includes a three-dimensional grid of gridblocks that represents a portion of the hydrocarbon reservoir. In some embodiments, the method further includes determining that the portion of the hydrocarbon reservoir represented by the gridblocks has mixed wettability, where the generation of the divided model is conducted in response to determining that the portion of the hydrocarbon reservoir represented by the gridblocks has mixed wettability. In certain embodiments, the method further includes, for each pair of a water-wet gridblock and an oil-wet gridblock generated from a gridblock, determining a transmissibility multiplier that corresponds to a degree of connectivity between the water-wet gridblock and the oil-wet gridblock, where the divided reservoir model defines the transmissibility multiplier for each pair of a water-wet gridblock and an oil-wet gridblock. In some embodiments, the method further includes generating, based on the simulation of the hydrocarbon reservoir, a field development plan (FDP) for the hydrocarbon reservoir. In certain embodiments, the method further includes: identifying, based on the simulation of the hydrocarbon reservoir, well drilling parameters; and drilling, based on the well drilling parameters, a well in the hydrocarbon reservoir. In some embodiments, the method further includes: identifying, based on the simulation of the hydrocarbon reservoir, well operating parameters; and operating, based on the well operating parameters, a well in the hydrocarbon reservoir.

Provided in some embodiments is a hydrocarbon reservoir development system that includes: a hydrocarbon reservoir control system adapted to perform the following operations: determine a reservoir model of a hydrocarbon reservoir that defines gridblocks that each represent a respective portion of the hydrocarbon reservoir and columns of the gridblocks that each represent a vertical segment of the hydrocarbon reservoir; acquire nano-images of a rock sample acquired from the hydrocarbon reservoir; determine, based on the nano-images of the rock sample, properties of an inorganic pore network of the rock sample and properties of an organic pore network of the rock sample; generate a divided reservoir model of the hydrocarbon reservoir that is representative of the inorganic and organic pore networks of the hydrocarbon reservoir, the generating of the divided reservoir model including: for each of the columns of gridblocks, divide each of the gridblocks of the column into: a water-wet gridblock associated with the properties of the inorganic pore network determined based on the nano-images of the rock sample; and an oil-wet gridblock associated with the properties of the organic pore network determined based on the nano-images of the rock sample; and generate, using the divided reservoir model of the hydrocarbon reservoir, a simulation of the hydrocarbon reservoir.

In some embodiments, acquiring nano-images of the rock sample includes conducting Focused Ion Beam (FIB) or Scanning Electron Microscopy (SEM) imaging of the rock sample to acquire FIB or SEM images of the rock sample. In certain embodiments, the reservoir model includes a three-dimensional grid of gridblocks that represents a portion of the hydrocarbon reservoir. In some embodiments, the method further includes determining that the portion of the hydrocarbon reservoir represented by the gridblocks has mixed wettability, and where the generation of the divided model is conducted in response to determining that the portion of the hydrocarbon reservoir represented by the gridblocks has mixed wettability. In certain embodiments, the method further includes, for each pair of a water-wet gridblock and an oil-wet gridblock generated from a gridblock, determining a transmissibility multiplier that corresponds to a degree of connectivity between the water-wet gridblock and the oil-wet gridblock, and where the divided reservoir model defines the transmissibility multiplier for each pair of a water-wet gridblock and an oil-wet gridblock. In some embodiments, the method further includes generating, based on the simulation of the hydrocarbon reservoir, a field development plan (FDP) for the hydrocarbon reservoir. In certain embodiments, the method further includes: identifying, based on the simulation of the hydrocarbon reservoir, well drilling parameters; and drilling, based on the well drilling parameters, a well in the hydrocarbon reservoir. In some embodiments, the method further includes: identifying, based on the simulation of the hydrocarbon reservoir, well operating parameters; and operating, based on the well operating parameters, a well in the hydrocarbon reservoir.

### BRIEF DESCRIPTION OF THE DRAWINGS

FIG. 1 is diagram that illustrates a hydrocarbon reservoir environment in accordance with one or more embodiments.

FIG. 2 is a flowchart that illustrates an example method of hydrocarbon reservoir modeling, simulation and development in accordance with one or more embodiments.

FIG. 3 is a diagram that illustrates a model representing of a portion of a hydrocarbon reservoir in accordance with one or more embodiments.

FIG. 4 is a diagram that illustrates a divided column representing of a portion of a model of a hydrocarbon reservoir in accordance with one or more embodiments.

FIG. 5 is a diagram that illustrates a divided model representing a portion of the hydrocarbon reservoir in accordance with one or more embodiments.

FIG. 6 is a diagram that illustrates an example computer system in accordance with one or more embodiments.

While this disclosure is susceptible to various modifications and alternative forms, specific embodiments are shown by way of example in the drawings and will be described in detail. The drawings may not be to scale. It should be understood that the drawings and the detailed descriptions are not intended to limit the disclosure to the particular form disclosed, but are intended to disclose modifications, equivalents, and alternatives falling within the spirit and scope of the present disclosure as defined by the claims.

### DETAILED DESCRIPTION

Described are embodiments of systems and methods for image-based modeling of rock having mixed wettability values, such as tight source rock. In some embodiments, gridblocks of a reservoir model for rock having mixed wettability values, such as unconventional shale, are divided into two parts—(1) an “oil-wet” gridblock that represents an organic (or “oil-wet”) pore network, and (2) a “water-wet” gridblock that represents an inorganic (or “water-wet”) pore network. In some embodiments, the properties of the oil-wet gridblocks and water wet gridblocks (for example, the porosity, the permeability, and the wettability of both networks, as well as the mutual connectivity of the two networks) are derived from nano-images of the rock. This may enable linking nano-image-derived rock properties to field-scale recovery behaviors that are observed by way of simulation of a model containing “wettability” divided gridblocks. In some embodiments, modeling of a “mixed wettability” reservoir, such as a reservoir formed of unconventional shale or other tight source rocks, includes the following: (1) acquiring a sample of rock from the reservoir (for example, by way of a coring operation); (2) acquiring three-dimensional (3D) nano-images of the rock that capture an inner pore structure of the rock (for example, using Focused Ion Beam (FIB) and Scanning Electron Microscopy (SEM)); (3) determining (based on the nano-images of the rock) for each of (a) an inorganic pore network in the rock, and (b) an organic pore network in the rock, the following properties of the rock: porosity, permeability, pore-size distribution, wettability, degree of connectivity, amount of oil, amount of gas, and amount of water; (4) generating a “divided model” of the reservoir comprising, for each column of gridblocks of a model of the reservoir, dividing the gridblocks to generate two “divided block columns” that include the following: (a) a first/inorganic divided block column having gridblocks associated with corresponding properties of the inorganic pore network in the rock; (b) a second/organic divided block column having gridblocks associated with corresponding properties of the organic pore network in the rock; and (c) for each pair of neighboring grid blocks of the two divided block columns, a transmissibility multiplier that corresponds to the degree of connectivity determined; and (5) simulating the reservoir using the divided model.

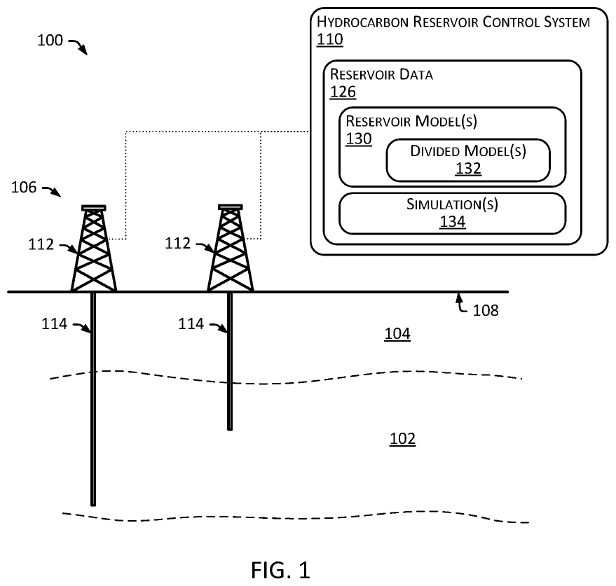

FIG. 1 is a diagram that illustrates a hydrocarbon reservoir environment (“reservoir environment”) 100 in accordance with one or more embodiments. In the illustrated embodiment, the reservoir environment 100 includes a hydrocarbon reservoir (“reservoir”) 102 located in a subsurface formation (“formation”) 104, and a hydrocarbon reservoir development system 106.

The formation 104 may include a porous or fractured rock formation that resides underground, beneath the Earth's surface (“surface”) 108. The reservoir 102 may include a portion of the formation 104 that contains (or that is determined to contain or expected to contain) a subsurface accumulation of hydrocarbons, such as oil and gas. The formation 104 and the reservoir 102 may each include different layers of rock having varying characteristics, such as varying degrees of permeability, porosity or fluid saturations. The hydrocarbon reservoir development system 106 may facilitate the extraction (or “production”) of hydrocarbons from the reservoir 102.

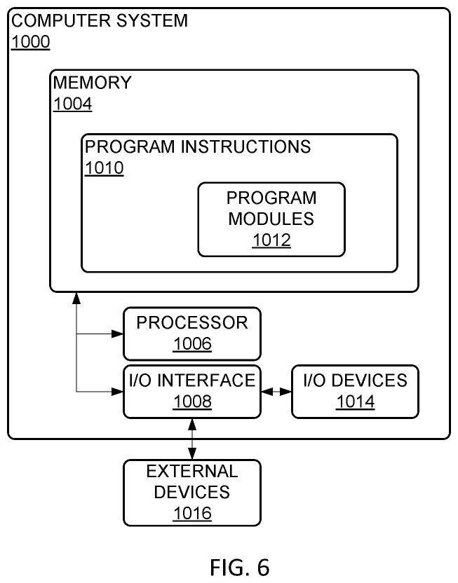

In some embodiments, the hydrocarbon reservoir development system 106 includes a hydrocarbon reservoir control system (“control system”) 110 and one or more wells 112. In some embodiments, the control system 110 includes a computer system that is the same as or similar to that of computer system 1000 described with regard to at least FIG. 6. Each of the wells 112 may be defined by a wellbore 114 that extends from the surface 108 into a target zone of the formation 104, such as the reservoir 102. The wellbore 114 may be created, for example, by a drill bit boring along a path (or “trajectory”) through the formation 104 and the reservoir 102. In some embodiment, the hydrocarbon reservoir development system 106 includes a drilling system. The drilling system may be operable to drill the wellbores 114 of the wells 112, or conduct coring operations to extract rock samples (or “cores”) from the reservoir 102 by way of the wellbores 114.

In some embodiments, the control system 110 controls operations for developing the reservoir 102. For example, the control system 110 may control logging operations used to acquire data for the reservoir 102, and may control processing that generates models and simulations (for example, based on the data acquired) that characterize the reservoir 102. In some embodiments, the control system 110 determines drilling parameters or operating parameters for the wells 112 in the reservoir 102, or controls drilling or operating of the wells 112 in accordance with drilling or operating parameters. This can include, for example, the control system 110 determining drilling parameters (for example, determining well locations and trajectories) for the reservoir 102, controlling drilling of the wells 112 in accordance with the drilling parameters (for example, controlling a well drilling system of the hydrocarbon reservoir development system 106 to drill the wells 112 at the well locations and following the trajectories), determining operating parameters (for example, determining production rates and pressures for “production” wells 112 and injection rates and pressure for “injection” wells 112), and controlling operations of the wells 112 in accordance with the operating parameters (for example, controlling a well operating system of the hydrocarbon reservoir development system 106 to operate the production wells 112 to produce hydrocarbons from the reservoir 102 in accordance with the production rates and pressures determined for the respective wells 112, and controlling the injection wells 112 to inject substances, such as water, into the reservoir 102 in accordance with the injection rates and pressures determined for the respective wells 112). In some embodiments, the control system 110 determines monitoring parameters or controls operations of “monitoring” wells 112 accordingly. For example, the control system 110 may determine wellbore logging parameters for monitoring wells 112, and control logging tools and sensors within the wellbores 114 of the monitoring wells 112 in accordance with the wellbore logging parameters for the respective monitoring wells 112.

In some embodiments, the control system 110 stores in a memory, or otherwise has access to, reservoir data 126. The reservoir data 126 may include data that is indicative of properties of the reservoir 102. In some embodiments, the reservoir data 126 includes one or more models 130 of the reservoir 102, or one or more simulations 134 of the reservoir 102.

A model 130 of the reservoir 102 may include a three-dimensional (3D) grid of cells representing a portion of the reservoir 102. Each of the cells may include a volume (for example, a cuboid shaped cell, or “gridblock”) that represents a corresponding volume within the reservoir 102, and may be associated with properties of the corresponding volume within the reservoir 102. This can include properties of the volume itself, such as a fluid saturation or porosity of the volume within the reservoir 102, as well as interfaces with adjacent cells, such as fluxes representing a fluid interchange between the cell and respective ones of the other cells directly adjacent the cell (for example, between shared faces of adjacent cells). The properties of each of the cells may be determined based on data acquired for the reservoir 102, such as seismic logs of the reservoir 102, downhole logs of wells already drilled into the reservoir 102, data acquired from core samples extracted from the reservoir 102, or recorded data for reservoirs having characteristics similar to those of the reservoir 102.

A simulation of the reservoir 102 (or “reservoir simulation”) may be data that includes a prediction of movement of fluids, such as water or hydrocarbons, within the reservoir 102 over time. In some embodiments, a simulation 134 of the reservoir 102 is generated based on a model 130 of the reservoir 102. For example, a simulation 134 may include a snapshot of where fluid is expected to be within the reservoir 102 one year from now based on a model 130 that indicates present characteristics of the reservoir 102 (such as the current location of water and hydrocarbons in the reservoir 102) and expected operating conditions for the wells 112 in the reservoir 102 over the next year (such as operating parameters for wells 112 in the reservoir 102 over the next year defined in an FDP). In some embodiments, a simulation 134 includes a sequence of snapshots over time that demonstrates the predicted movement/location of the fluids within the reservoir 102 at different points in time (for example, the predicted location of fluids at year one, at year two, and at year three). Simulations 134 may be used as a basis for develop the reservoir 102. For example, a simulation 134 of the reservoir 102 may be used to determine drilling or operating parameters for wells 112 in the reservoir 102.

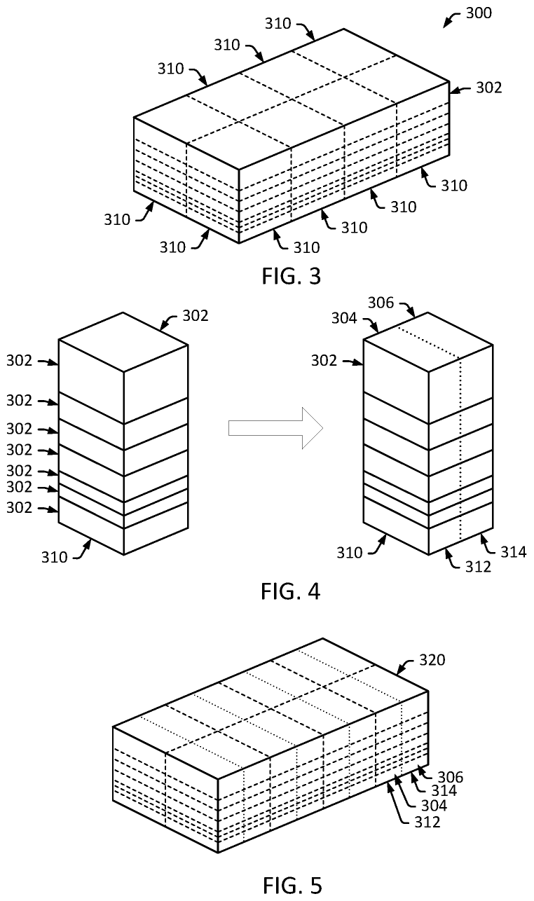

In some embodiments, a model 130 of the reservoir 102 is processed to generate one or more simulations 134 of the reservoir 102. For example, a model 130 may be generated that includes a 3D grid of cells and corresponding properties, which can be processed to generate a simulation 134 of the reservoir 102. The 3D grid may, for example, include individual gridblocks that each represent a respective portion of the reservoir 102 and that, together, represent a larger portion of the reservoir 102. Referring to an example illustrated in FIG. 3, a portion of the reservoir 102 may be represented by a 3D grid 300 formed of fifty-six individual gridblocks 302 that each represent a respective portion of the reservoir 102 and that, together, represent a larger portion of the reservoir 102. In some embodiments, each of the gridblocks of a 3D grid is associated with properties that correspond to the portion of the reservoir 102 represented by the gridblock. For example, each of the gridblocks 302 of the 3D grid 300 of FIG. 3 may be associated with a given set of properties, such as porosity, permeability, pore-size distribution, wettability, degree of connectivity, amount of oil, amount of gas, and amount of water, that correspond to the physical volume of the reservoir 102 represented by the gridblock 302. In some embodiments, properties associated with the gridblocks are determined based on nano-images of rock samples extracted from the reservoir 102. For example, a rock sample (or “core”) may be extracted from the reservoir 102 by way of a coring operation and be transported to a core laboratory where it is physically assessed to determine properties of the rock forming the reservoir 102. The assessment may include, for example, using FIB imaging or SEM imaging to acquire 3D FIB or SEM nano-images of the rock that capture an inner pore structure of the rock. Given the detail captured in the nano-images, the nano-images may be used to accurately determine the porosity, the permeability, and the wettability of both of inorganic (or “water-wet”) and organic (or “oil-wet”) pore networks contained in the rock sample, as well as a measure of a degree of connectivity between the two networks. For example, determining properties of formation rock may include generating a 3D image of a portion of sample of formation rock that is used to determine properties of the rock forming the reservoir 102. This may include, for example, (1) isolating a portion of a core for testing (a “test sample”) (e.g., extracting a small volume of the core for testing); (2) for each of multiple layers of the test sample: (a) conducting surface imaging of an exposed surface of the layer of the test sample (e.g., scanning a surface of the test sample using SEM) to generate a respective layer image; and (b) milling a thin layer off of the surface of the currently exposed surface/layer of the test sample (e.g., using FIB milling) to expose a next “deeper” surface/layer of the test sample to be imaged, to generate a set of layer images for the layers; (3) conducting segmenting of the set of layer images that includes: (a) dividing each of the layer images into a grid of grid cells; and (b) assigning an attribute to each of the grid cells (e.g., attributes of pores, rock, grain or other elements) based on the SEM results; and (4) combining the grid cells of the various layers to generate a 3D image of the test sample. Characteristics of the test sample may be determined based on the attributes incorporated into the 3D image. For example, porosity may be ascertained from a 3D image by summing the pore volume in the 3D image to determine a total pore volume and dividing the total pore volume by the total volume of the 3D image. Examples of 3D images and techniques for determining properties, such as permeability, by way of computer-simulated flow studies of the pore-space geometry of a 3D image are described in Shabro, V., S. Kelly, C. Torres-Verdin & K. Sepehrnoori: “Pore-Scale Modeling Of Electrical Resistivity And Permeability In Fib-Sem Images Of Hydrocarbon-Bearing Shale,” 54th SPWLA Annual Logging Symposium, Society of Petrophysicists and Well Log Analysts, New Orleans (2013), which is hereby incorporated by reference. Such techniques can be used in place of traditional testing that can be complex and lengthy. For example, in the case of ultra-low-permeability rock, such as shales, it can be difficult to measure these quantities by conventional means, such as flow-through core sample laboratory tests, that can take months and require management of large pressure gradients and relatively small flow rates that can be too small to measure accurately. Thus, such imaged-based determination of properties can be useful for determining properties, such as porosity and permeability.

In some embodiments, gridblocks of a model 130 for rock having mixed wettability values, such as gridblocks representative of portion of the reservoir 102 formed of unconventional shale, are divided into two parts—(1) an “oil-wet” gridblock that represents an organic (or “oil-wet”) pore network, and (2) a “water-wet” gridblock that represents an inorganic (or “water-wet”) pore network. For example, referring to FIGS. 3-5, each of the gridblocks 302 of the “undivided” 3D grid 300 of FIG. 3 (which includes columns of gridblocks 310 that each represent a vertical segment of the reservoir 102) may be associated with a mixed wettability value and be divided into an “oil-wet” gridblock 304 and a “water-wet” gridblock 306, as illustrated in FIG. 4. As a result, each column of gridblocks 310 may be divided into two associated columns of gridblocks 310, including an organic (or “oil-wet”) column of gridblocks 312 and an inorganic (or “water-wet”) column of gridblocks 314. Each of the oil-wet gridblocks 304 (and the organic (or “oil-wet”) column of gridblocks 312) may be associated with properties of the organic (or “oil-wet”) pore network, and each of the “water-wet” gridblocks 306 (and the inorganic (or “water-wet”) column of gridblocks 314) may be associated with properties of the inorganic (or “water-wet”) pore network. In some embodiments, each pair of an oil-wet gridblock 304 and a water-wet gridblock 306 generated from a given gridblock 302 may be associated with a transmissibility multiplier that corresponds to the degree of connectivity between the oil-wet and water-wet pore networks of the given gridblock 302. Accordingly, an “undivided” model 130 (for example, represented by the “undivided” 3D grid 300 of FIG. 3) may be transformed into a “divided” model 132 (for example, represented by the “divided” 3D grid 320 of FIG. 5) defining “divided” gridblocks (and divided columns of gridblocks) that provides an enhanced representation of the reservoir 102. Such a divided model 132 may be employed in a simulation operation to generate a “dual pore network” enhanced simulation 134 of the reservoir 102 that takes into account the representations of the oil-wet and water-wet pore networks of the reservoir 102.

In some embodiments, modeling of the reservoir 102 includes the following: (1) acquiring a sample of rock from the reservoir 102 (for example, by way of a coring operation); (2) acquiring 3D nano-images of the rock that capture an inner pore structure of the rock (for example, using FIB and SEM imaging); (3) determining (based on the nano-images of the rock) for each of (a) an inorganic pore network in the rock, and (b) an organic pore network in the rock, the following properties of the rock: porosity, permeability, pore-size distribution, wettability, degree of connectivity, amount of oil, amount of gas, and amount of water; (4) generating a divided model 132 of the reservoir 102 including, for each column of gridblocks of the model 130 of the reservoir 102 (for example, for each column of gridblocks 310), dividing the gridblocks to generate two “divided block columns” that include the following: (a) a first/inorganic divided block column having gridblocks associated with corresponding properties of the inorganic pore network in the rock (for example, an inorganic (or “water-wet”) column of gridblocks 314); (b) a second/organic divided block column having gridblocks associated with corresponding properties of the organic pore network in the rock (for example, an organic (or “oil-wet”) column of gridblocks 312); and (c) for each pair of neighboring grid blocks of the two divided block columns (for example, for each pair of an oil-wet gridblock 304 and a water-wet gridblock 306 generated from a given gridblock 302), a transmissibility multiplier that corresponds to the degree of connectivity determined; and (5) simulating the reservoir 102 using the divided model 132 to generate a simulation 134 of the reservoir 102, which may be used to estimate performance of the reservoir 102 or as a basis for operating and developing the reservoir 102.

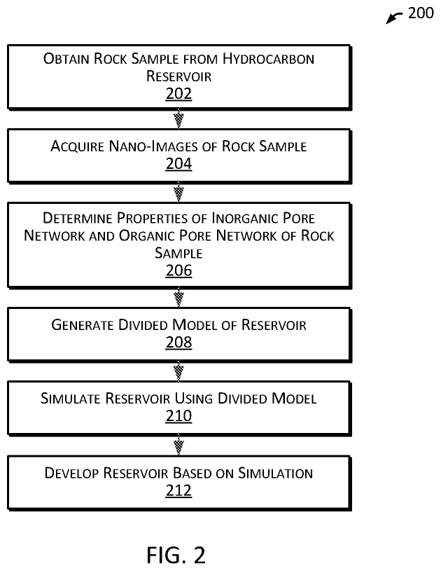

FIG. 2 is a diagram that illustrates a method 200 of developing a hydrocarbon reservoir in accordance with one or more embodiments. Some or all of the procedural elements of the method 200 may be performed, for example, by the control system 110 or another reservoir operator.

In some embodiments, the method 200 includes obtaining a rock sample from a hydrocarbon reservoir (block 202). Obtaining a rock sample from a hydrocarbon reservoir may include extracting a rock sample (or “core”) from a hydrocarbon reservoir. For example, obtaining a rock sample from a hydrocarbon reservoir may include the control system 110 controlling the system 106 to conduct a coring operation to extract one or more rock samples from the reservoir 102. The one or more rock samples may, for example, be transported to a core laboratory for assessment. The assessment may include acquiring and interpreting nano-images of the rock sample to determine properties of inorganic and organic pore networks of the rock sample.

In some embodiments, the method 200 includes acquiring nano-images of the rock sample (block 204). Acquiring nano-images of the rock sample may include conducting imaging operations, such as FIB or SEM imaging operations, to acquire images that are indicative of properties of inorganic and organic pore networks present in the rock sample. For example, acquiring nano-images of the rock sample may include the control system 110 controlling a core sample assessment in a core laboratory to include conduct a FIB scan or a SEM scan of the rock sample from the reservoir 102. This may generate corresponding FIB and SEM nano-images of the rock sample, which capture visual aspects of the rock sample that are indicative of properties of inorganic and organic pore networks present in the rock sample (and at a corresponding location in the reservoir 102 from which the rock sample was extracted). In some embodiments, multiple samples may be extracted and imaged to generate multiple nano-images that are indicative of properties of inorganic and organic pore networks present in the rock samples (and at a corresponding locations in the reservoir 102 from which the rock samples were extracted).

In some embodiments, the method 200 includes determining properties of the inorganic and organic pore networks of the rock sample (block 206). Determining properties of inorganic and organic pore networks in the rock sample may include determining the following, based on nano-images of the rock sample: (1) that both inorganic and organic pore networks are present in the rock sample, and (2) for each of (a) the inorganic pore network in the rock sample and (b) the organic pore network in the rock, some or all of the following properties of the rock sample: porosity, permeability, pore-size distribution, wettability, degree of connectivity, amount of oil, amount of gas, and amount of water. Porosity may be a measure of an amount of open space within the rock sample. Permeability may be a measure of the ease with which a fluid can move through the rock sample. Pore-size distribution may represent the relative abundance of each pore size within the rock sample. Wettability may be a measure of the tendency of one fluid to spread on, or adhere to, a solid surface in the presence of other immiscible fluids. A water-wet rock surface may be a surface of rock that has a strong preference to be coated, or “wetted,” by the water phase, so that there will be a continuous water phase on the rock surfaces. An oil-wet rock surface may be a surface of rock that has a strong preference to be coated, or “wetted,” by the oil phase, so that there will be a continuous oil phase on the rock surfaces. Degree of connectivity may be as low as zero (e.g., indicating no connections between the oil-wet and water-wet pore networks) with complete connection at the upper limit (e.g., with 1 being completely connected and 0 being completely disconnected). The transmissibility multiplier may be used to account for a reduction from the completely connected case. This may be ascertained, for example, from analysis of both pore-network geometries using known methods. Amount of oil, gas or water may be a measure of an amount of oil, gas or water, respectively, contained in the rock sample. Some or all of the properties may be determined by way of assessment of the physical rock sample in a core laboratory.

In some embodiments, the method 200 includes generating a divided model of the reservoir (block 208). Generating a divided model of the reservoir may include generating a model of the reservoir that includes gridblocks that distinctly represent properties of the inorganic pore network in the rock sample or the organic pore network in the rock sample. For example, generating a divided model of the reservoir 102 may include generating a divided model 132 of the reservoir 102 that includes oil-wet gridblocks 304 and water-wet gridblocks 306 that distinctly represent respective properties of the organic and inorganic pore networks in the rock sample. Referring to FIGS. 3-5, this may include, for example, dividing each column of gridblocks 310 (which each represent a respective vertical segment of the reservoir 102) of the 3D grid 300 of FIG. 3 into two associated columns of gridblocks 310, including an organic (or “oil-wet”) column of gridblocks 312 and an inorganic (or “water-wet”) column of gridblocks 314 (for example, as illustrated in FIG. 4) to generate the divided 3D grid 320 of FIG. 5 that forms the basis of the divided model 132. The oil-wet gridblocks 304 (and the organic (or “oil-wet”) column of gridblocks 312) may be associated with the determined properties of the organic (or “oil-wet”) pore network. The “water-wet” gridblocks 306 (and the inorganic (or “water-wet”) column of gridblocks 314) may be associated with the determined properties of the inorganic (or “water-wet”) pore network. Each pair of an oil-wet gridblock 304 and a water-wet gridblock 306 generated from a given gridblock 302 may be associated with a transmissibility multiplier that corresponds to the determined degree of connectivity between the oil-wet and water-wet pore networks of the given gridblock 302. Thus, each gridblock 302 may be divided into a pair of “sub” gridblocks, including an oil-wet gridblock 304 associated with the determined properties of the organic (or “oil-wet”) pore network and a water-wet gridblock 306 associated with the determined properties of the inorganic (or “water-wet”) pore network, sharing an association with a transmissibility multiplier that corresponds to a determined degree of connectivity between the two “sub” gridblocks 304 and 306. The pore network properties are determined from nano-images of a given core, then associated with a single block, multiple blocks or all blocks. This may be accomplished, for example, by way of core data populating of simulation models. In some embodiments, it may be determined that a portion of the hydrocarbon reservoir 102 represented by gridblocks of a model 130 has “mixed wettability” responsive to the determination that both inorganic and organic pore networks are present in a rock sample from the reservoir 102, and the divided model 132 may be generated in response to the determination that a portion of the hydrocarbon reservoir 102 represented by gridblocks of a model 130 has mixed wettability.

In some embodiments, the method 200 includes simulating the reservoir using the divided model (block 210). Simulating of the reservoir using the divided model may include processing a divided model of the reservoir to generate a simulation the reservoir. For example, simulating the reservoir 102 using the divided model 132 may include the control system 110 processing the divided model 132 to generate a simulation 134 the reservoir 102. The simulation 134 may include a prediction of movement of fluids, such as water or hydrocarbons, over time within the portion of the reservoir 102 represented by the 3D divided grid of the divided model 132. For example, a 1-year simulation of the reservoir 102 based on the divided model 132 representing the characteristics of the reservoir on Jan. 1, 2019 may include a prediction of where fluids, such as water or hydrocarbons, will be located within the portion of the reservoir 102 represented by the 3D divided grid of the divided model 132, on Jan. 1, 2020. In some embodiments, the divided model 132 or the simulation 134 is presented on a graphical display for viewing, for example, by a reservoir operator.

In some embodiments, the method 200 includes developing the reservoir based on the simulation of the reservoir (block 212). Developing the reservoir based on the simulation of the reservoir may include defining or conducting various operations for development of the reservoir based on the simulation of the reservoir. For example, developing the reservoir 102 based on a simulation 134 of the reservoir 102 may include the control system 110 or (another operator of the reservoir 102) determining drilling parameters or operating parameters for wells 112 in the reservoir 102, or controlling drilling or operating of the wells 112 in accordance with the drilling or operating parameters. In some embodiments, an FDP may be generated for the reservoir 102 based on the on a simulation 134 of the reservoir 102. For example, the control system 110 or (another operator of the reservoir 102) may generate an FDP that specifies parameters for developing the reservoir 102, such as the drilling parameters or operating parameters for wells 112 in the reservoir 102 in 2019, based on the on a simulation 134 that indicates predicted fluid movement in the portion of the reservoir 102 represented by the model 130 over the timespan from Jan. 1, 2019 to Jan. 1, 2020. In such an embodiment, the reservoir 102 may be developed in accordance with the FDP.

FIG. 10 is a diagram that illustrates an example computer system (or “system”) 1000 in accordance with one or more embodiments. The system 1000 may include a memory 1004, a processor 1006 and an input/output (I/O) interface 1008. The memory 1004 may include non-volatile memory (for example, flash memory, read-only memory (ROM), programmable read-only memory (PROM), erasable programmable read-only memory (EPROM), electrically erasable programmable read-only memory (EEPROM)), volatile memory (for example, random access memory (RAM), static random access memory (SRAM), synchronous dynamic RAM (SDRAM)), or bulk storage memory (for example, CD-ROM or DVD-ROM, hard drives). The memory 1004 may include a non-transitory computer-readable storage medium having program instructions 1010 stored on the medium. The program instructions 1010 may include program modules 1012 that are executable by a computer processor (for example, the processor 1006) to cause the functional operations described, such as those described with regard to the control system 110 or the method 200.

The processor 1006 may be any suitable processor capable of executing program instructions. The processor 1006 may include one or more processors that carry out program instructions (for example, the program instructions of the program modules 1012) to perform the arithmetical, logical, or input/output operations described. The processor 1006 may include multiple processors that can be grouped into one or more processing cores that each include a group of one or more processors that are used for executing the processing described here, such as the independent parallel processing of partitions (or “sectors”) by different processing cores to generate a simulation of a reservoir. The I/O interface 1008 may provide an interface for communication with one or more I/O devices 1014, such as a joystick, a computer mouse, a keyboard, or a display screen (for example, an electronic display for displaying a graphical user interface (GUI)). The I/O devices 1014 may include one or more of the user input devices. The I/O devices 1014 may be connected to the I/O interface 1008 by way of a wired connection (for example, an Industrial Ethernet connection) or a wireless connection (for example, a Wi-Fi connection). The I/O interface 1008 may provide an interface for communication with one or more external devices 1016, such as sensors, valves, pumps, motors, computers or communication networks. In some embodiments, the I/O interface 1008 includes an antenna or a transceiver.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the embodiments. It is to be understood that the forms of the embodiments shown and described here are to be taken as examples of embodiments. Elements and materials may be substituted for those illustrated and described here, parts and processes may be reversed or omitted, and certain features of the embodiments may be utilized independently, all as would be apparent to one skilled in the art after having the benefit of this description of the embodiments. Changes may be made in the elements described here without departing from the spirit and scope of the embodiments as described in the following claims. Headings used here are for organizational purposes only and are not meant to be used to limit the scope of the description.

It will be appreciated that the processes and methods described here are example embodiments of processes and methods that may be employed in accordance with the techniques described here. The processes and methods may be modified to facilitate variations of their implementation and use. The order of the processes and methods and the operations provided may be changed, and various elements may be added, reordered, combined, omitted, modified, and so forth. Portions of the processes and methods may be implemented in software, hardware, or a combination thereof. Some or all of the portions of the processes and methods may be implemented by one or more of the processors/modules/applications described here.

As used throughout this application, the word “may” is used in a permissive sense (meaning having the potential to), rather than the mandatory sense (meaning must). The words “include,” “including,” and “includes” mean including, but not limited to. As used throughout this application, the singular forms “a,” “an,” and “the” include plural referents unless the content clearly indicates otherwise. Thus, for example, reference to “an element” may include a combination of two or more elements. As used throughout this application, the term “or” is used in an inclusive sense, unless indicated otherwise. That is, a description of an element including A or B may refer to the element including one or both of A and B. As used throughout this application, the phrase “based on” does not limit the associated operation to being solely based on a particular item. Thus, for example, processing “based on” data A may include processing based at least in part on data A and based at least in part on data B, unless the content clearly indicates otherwise. As used throughout this application, the term “from” does not limit the associated operation to being directly from. Thus, for example, receiving an item “from” an entity may include receiving an item directly from the entity or indirectly from the entity (for example, by way of an intermediary entity). Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. In the context of this specification, a special purpose computer or a similar special purpose electronic processing/computing device is capable of manipulating or transforming signals, typically represented as physical, electronic or magnetic quantities within memories, registers, or other information storage devices, transmission devices, or display devices of the special purpose computer or similar special purpose electronic processing/computing device.

What is claimed is:

1. A method of developing a hydrocarbon reservoir, the method comprising:
determining a reservoir model of a hydrocarbon reservoir, the reservoir model defining gridblocks that each represent a respective portion of the hydrocarbon reservoir and columns of the gridblocks that each represent a vertical segment of the hydrocarbon reservoir;
acquiring nano-images of a rock sample acquired from the hydrocarbon reservoir;
determining, based on the nano-images of the rock sample, properties of an inorganic pore network of the rock sample and properties of an organic pore network of the rock sample;
generating a divided reservoir model of the hydrocarbon reservoir that is representative of the inorganic and organic pore networks of the hydrocarbon reservoir, the generating of the divided reservoir model comprising:
for each of the columns of gridblocks, dividing each of the gridblocks of the column into:
a water-wet gridblock associated with the properties of the inorganic pore network determined based on the nano-images of the rock sample; and
an oil-wet gridblock associated with the properties of the organic pore network determined based on the nano-images of the rock sample; and
generating, using the divided reservoir model of the hydrocarbon reservoir, a simulation of the hydrocarbon reservoir.

2. The method of claim 1, wherein acquiring nano-images of the rock sample comprises conducting Focused Ion Beam (FIB) or Scanning Electron Microscopy (SEM) imaging of the rock sample to acquire FIB or SEM images of the rock sample.

3. The method claim 1, wherein the reservoir model comprises a three-dimensional grid of gridblocks that represents a portion of the hydrocarbon reservoir.

4. The method of claim 1, further comprising determining that the portion of the hydrocarbon reservoir represented by the gridblocks has mixed wettability, wherein the generation of the divided model is conducted in response to determining that the portion of the hydrocarbon reservoir represented by the gridblocks has mixed wettability.

5. The method of claim 1, further comprising, for each pair of a water-wet gridblock and an oil-wet gridblock generated from a gridblock, determining a transmissibility multiplier that corresponds to a degree of connectivity between the water-wet gridblock and the oil-wet gridblock, wherein the divided reservoir model defines the transmissibility multiplier for each pair of a water-wet gridblock and an oil-wet gridblock.

6. The method of claim 1, further comprising generating, based on the simulation of the hydrocarbon reservoir, a field development plan (FDP) for the hydrocarbon reservoir.

7. The method of claim 1, further comprising:
identifying, based on the simulation of the hydrocarbon reservoir, well drilling parameters; and
drilling, based on the well drilling parameters, a well in the hydrocarbon reservoir.

8. The method of claim 1, further comprising:
identifying, based on the simulation of the hydrocarbon reservoir, well operating parameters; and
operating, based on the well operating parameters, a well in the hydrocarbon reservoir.

9. A non-transitory computer readable storage medium comprising program instructions stored thereon that are executable by a processor to perform the following operations for developing a hydrocarbon reservoir:
determining a reservoir model of a hydrocarbon reservoir, the reservoir model defining gridblocks that each represent a respective portion of the hydrocarbon reservoir and columns of the gridblocks that each represent a vertical segment of the hydrocarbon reservoir;
acquiring nano-images of a rock sample acquired from the hydrocarbon reservoir;
determining, based on the nano-images of the rock sample, properties of an inorganic pore network of the rock sample and properties of an organic pore network of the rock sample;
generating a divided reservoir model of the hydrocarbon reservoir that is representative of the inorganic and organic pore networks of the hydrocarbon reservoir, the generating of the divided reservoir model comprising:
for each of the columns of gridblocks, dividing each of the gridblocks of the column into:
a water-wet gridblock associated with the properties of the inorganic pore network determined based on the nano-images of the rock sample; and
an oil-wet gridblock associated with the properties of the organic pore network determined based on the nano-images of the rock sample; and
generating, using the divided reservoir model of the hydrocarbon reservoir, a simulation of the hydrocarbon reservoir.

10. The medium of claim 9, wherein acquiring nano-images of the rock sample comprises conducting Focused Ion Beam (FIB) or Scanning Electron Microscopy (SEM) imaging of the rock sample to acquire FIB or SEM images of the rock sample.

11. The medium of claim 9, wherein the reservoir model comprises a three-dimensional grid of gridblocks that represents a portion of the hydrocarbon reservoir.

12. The medium of claim 9, further comprising determining that the portion of the hydrocarbon reservoir represented by the gridblocks has mixed wettability, wherein the generation of the divided model is conducted in response to determining that the portion of the hydrocarbon reservoir represented by the gridblocks has mixed wettability.

13. The medium of claim 9, further comprising, for each pair of a water-wet gridblock and an oil-wet gridblock generated from a gridblock, determining a transmissibility multiplier that corresponds to a degree of connectivity between the water-wet gridblock and the oil-wet gridblock, wherein the divided reservoir model defines the transmissibility multiplier for each pair of a water-wet gridblock and an oil-wet gridblock.

14. The medium of claim 9, further comprising generating, based on the simulation of the hydrocarbon reservoir, a field development plan (FDP) for the hydrocarbon reservoir.

15. The medium of claim 9, further comprising:
identifying, based on the simulation of the hydrocarbon reservoir, well drilling parameters; and
drilling, based on the well drilling parameters, a well in the hydrocarbon reservoir.

16. The medium of claim 9, further comprising:
identifying, based on the simulation of the hydrocarbon reservoir, well operating parameters; and
operating, based on the well operating parameters, a well in the hydrocarbon reservoir.

17. A hydrocarbon reservoir development system comprising:
a hydrocarbon reservoir control system configured to perform the following operations:
determine a reservoir model of a hydrocarbon reservoir, the reservoir model defining gridblocks that each represent a respective portion of the hydrocarbon reservoir and columns of the gridblocks that each represent a vertical segment of the hydrocarbon reservoir;
acquire nano-images of a rock sample acquired from the hydrocarbon reservoir;
determine, based on the nano-images of the rock sample, properties of an inorganic pore network of the rock sample and properties of an organic pore network of the rock sample;
generate a divided reservoir model of the hydrocarbon reservoir that is representative of the inorganic and organic pore networks of the hydrocarbon reservoir, the generating of the divided reservoir model comprising:
for each of the columns of gridblocks, divide each of the gridblocks of the column into:
a water-wet gridblock associated with the properties of the inorganic pore network determined based on the nano-images of the rock sample; and
an oil-wet gridblock associated with the properties of the organic pore network determined based on the nano-images of the rock sample; and
generate, using the divided reservoir model of the hydrocarbon reservoir, a simulation of the hydrocarbon reservoir.

18. The system of claim 17, wherein acquiring nano-images of the rock sample comprises conducting Focused Ion Beam (FIB) or Scanning Electron Microscopy (SEM) imaging of the rock sample to acquire FIB or SEM images of the rock sample.

19. The system of claim 17, wherein the reservoir model comprises a three-dimensional grid of gridblocks that represents a portion of the hydrocarbon reservoir.

20. The system of claim 17, wherein the operations further comprise determining that the portion of the hydrocarbon reservoir represented by the gridblocks has mixed wettability, and wherein the generation of the divided model is conducted in response to determining that the portion of the hydrocarbon reservoir represented by the gridblocks has mixed wettability.

21. The system of claim 17, wherein the operations further comprise, for each pair of a water-wet gridblock and an oil-wet gridblock generated from a gridblock, determining a transmissibility multiplier that corresponds to a degree of connectivity between the water-wet gridblock and the oil-wet gridblock, and wherein the divided reservoir model defines the transmissibility multiplier for each pair of a water-wet gridblock and an oil-wet gridblock.

22. The system of claim 17, wherein the operations further comprise generating, based on the simulation of the hydrocarbon reservoir, a field development plan (FDP) for the hydrocarbon reservoir.

23. The system of claim 17, wherein the operations further comprise:
identifying, based on the simulation of the hydrocarbon reservoir, well drilling parameters; and
drilling, based on the well drilling parameters, a well in the hydrocarbon reservoir.

24. The system of claim 17, wherein the operations further comprise:
identifying, based on the simulation of the hydrocarbon reservoir, well operating parameters; and
operating, based on the well operating parameters, a well in the hydrocarbon reservoir.
